(19) United States
(12) Patent Application Publication
(10) Pub. No.: US 2008/0154102 A1
(43) Pub. Date: Jun. 26, 2008

(54) Intraoperative imaging methods

(76) Inventors: John V. Frangioni, Wayland, MA (US); Eichii Tanaka, Hokkaido (JP); Loren J. Borud, Newton, MA (US)

Correspondence Address: FISH & RICHARDSON PC, P.O. BOX 1022, MINNEAPOLIS, MN, 55440-1022, US

(21) Appl. No.: 11/825,257

(22) Filed: Jul. 3, 2007

Related U.S. Application Data
(60) Provisional application No. 60/818,398, filed on Jul. 3, 2006.

(51) Int. Cl. A61B 6/00

(52) U.S. Cl. 600/317

(57) ABSTRACT

Described are methods for intraoperative imaging of anatomical structures using fluorescent compounds, e.g., compounds that fluoresce in the invisible light (IL) region of the spectrum, i.e., above 670 nm. An exemplary compound is methylene blue.

### CLAIM OF PRIORITY

This application claims the benefit of U.S. Provisional Patent Application Ser. No. 60/818,398, filed on Jul. 3, 2006, the entire contents of which are hereby incorporated by reference.

### FEDERALLY SPONSORED RESEARCH OR DEVELOPMENT

This invention was made with Government support under Grant No. R01-CA-115296 awarded by the National Institutes of Health and Grant No. DEFG02-01ER63188 awarded by the Department of Energy. The Government has certain rights in the invention.

### TECHNICAL FIELD

This invention relates to methods for intraoperative imaging using fluorescent compounds, e.g., compounds that fluoresce in the invisible light (IL) region of the spectrum, i.e., above 670 nm.

### BACKGROUND

During surgical procedures, it is often difficult for the surgeon to determine the location of structures such as the thoracic duct, urinary tract, and biliary tree, and perforating arteries.

### SUMMARY

The present invention is based, at least in part, on the discovery that certain dyes that fluoresce in the invisible light (IL) region of the spectrum are useful for imaging certain anatomical structures, e.g., during surgery. Thus, provided herein are methods for imaging structures such as the thoracic duct, urinary tract, biliary tree, and perforating arteries, using IL dyes.

In a first aspect, the invention features methods for imaging an anatomical structure selected from the group consisting of a biliary tree, a ureter, or a portion thereof, in vivo. The methods include administering to a subject a preparation including an invisible light (IL) fluorophore that is filtered by the kidney into the urine stream, filtered by the liver into the bile stream, or both, and obtaining an image of IL wavelength emissions from the fluorophore, wherein the image is a representation of the anatomical structure.

In another aspect, the invention features methods for imaging a thoracic duct or a portion thereof in vivo. The methods include injecting a preparation including an invisible light fluorophore (ILF) into a lymph node of a subject such that it appears in the thoracic duct, e.g., any lymph node that drains into the thoracic duct, e.g., at or below the level of the diaphragm, and obtaining an image of invisible light (IL) wavelength emissions, wherein the image is a representation of the thoracic duct. In some embodiments, a sufficient amount of time is allowed to pass for the ILF to flow into the thoracic duct before the image is obtained.

In some embodiments, the image includes some portion of the subject, and the image includes a first image obtained from one or more wavelengths of visible light and a second image obtained from IL wavelength emissions of the ILF. In some embodiments, the image includes a visible light image of the surgical field and an IL wavelength image of the surgical field. In some embodiments, the visible light image and the IL wavelength image are obtained concurrently.

In some embodiments, obtaining an image includes positioning an electronic imaging device, e.g., as described herein, adjacent to the structure; either the structure or the device can be moved so that the two are in close enough proximity to enable the imaging.

In some embodiments, the preparation is administered systemically, e.g., intravenously, or alternatively, by direct injection. The administration is not topical. In some embodiments, the anatomical structure is a biliary tree, and the preparation is administered by injection (e.g., cannulation) into a portal vein, left hepatic duct, or right hepatic duct. In some embodiments, the anatomical structure is a ureter, and the preparation is administered by injection into a renal artery, bladder, or ureter. In some embodiments, the methods include administration of a urinary alkalinizer, e.g., acetazolamide or sodium bicarbonate.

In some embodiments, the ILF is a near-infrared fluorophore (NIRF) with an emission wavelength in a range from about 670 nm to about 1,000 nm, e.g., IRDye78, IRDye80, IRDye38, IRDye40, IRDye41, IRDye700, IRDye800, Cy5.5, and Cy7, or an analog thereof.

In some embodiments, the near-infrared fluorophore is methylene blue (MB). In some embodiments, the preparation is a solution comprising about 100 nM-100 μM MB. In some embodiments, the methods include administering a sufficient amount of MB to achieve a concentration of about 10-40 μM MB in the structure to be imaged. In some embodiments, the MB is administered in a total systemic dose of about 0.1 to 10 mg/kg of body weight. In some embodiments, the MB is administered in a total systemic dose of about 1 mg/kg of body weight. In some embodiments, the MB is administered in a total systemic dose of less than 10 mg/kg, e.g., less than 7.5 mg/kg, of body weight.

In another aspect, the invention provides methods for imaging an anatomical structure, e.g., vasculature, biliary tree, thoracic duct, ureters, heart myocardium, a parathyroid gland, a pancreas (e.g., islet cells), a tumor (e.g., tumors of pancreatic or parathyroid origin), or a portion thereof, in vivo. The methods include systemically (e.g., intravenously) administering to a subject a preparation including methylene blue (MB) such that it appears in (i.e., flows into, e.g., by retrograde or anterograde flow) the anatomical structure, and obtaining an image of fluorescent emissions from the MB, wherein the image is a representation of the anatomical structure. In some embodiments, the tissue or organ has high uptake of MB, such as heart myocardium or the parathyroid glands.

In a further aspect, the invention provides methods for imaging first and second anatomical structures in vivo. The methods include administering, e.g., systemically administering (e.g., intravenously), to a subject a preparation including methylene blue (MB) such that it appears in (i.e., flows into, e.g., by retrograde or anterograde flow) the first anatomical structure, obtaining a first image of invisible light wavelength emissions of the MB, wherein the image is a representation of the first anatomical structure; administering to the subject a preparation including a second invisible light fluorophore (ILF) with an emission wavelength of at least about 780 nm, e.g., about 800 nm, such that the second ILF appears in (i.e., flows into, e.g., by retrograde or anterograde flow) the second anatomical structure; and obtaining a second image of the invisible light emissions of the second ILF, wherein the image of the invisible light emissions of the second ILF is a representation of a second anatomical structure.

In some embodiments, the anatomical structure represented by the first image is a portion of the vasculature, a biliary tree, a thoracic duct, a ureter, the heart, a parathyroid gland, a pancreas (e.g., islet cells), a tumor (e.g., a tumor of pancreatic or parathyroid origin), or a portion thereof. In some embodiments, the anatomical structure represented by the second image is a portion of the vasculature, e.g., a biliary tree, a thoracic duct, a ureter, a heart, a parathyroid gland, a pancreas (e.g., islet cells), a tumor (e.g., a tumor of pancreatic or parathyroid origin), or a portion thereof, and is different from the anatomical structure represented by the first image.

In some embodiments, the second ILF is indocyanine green (ICG) or a carboxylic acid of IRDye™800CW.

In some embodiments, the methods further include obtaining a visible light image of the structures. The first image, second image, and visible light image, can all be obtained concurrently, and optionally superimposed to create a merged image.

In an additional aspect, the invention features methods for imaging a portion of the vasculature in vivo. The methods include administering to a subject a preparation including methylene blue (MB) such that it appears in (i.e., flows into, e.g., by retrograde or anterograde flow) the vasculature, and obtaining an image of invisible light (IL) wavelength emissions, wherein the image is a representation of the vasculature.

In a further aspect, the invention provides methods for imaging the vasculature of a tissue for evaluation of flap design desirability, assessment of flap viability, or determination of suitability of a failing flap for salvage therapy with fibrinolytics. The methods include administering to the subject a preparation including an invisible light (IL) fluorophore such that it appears in (i.e., flows into, e.g., by retrograde or anterograde flow) the vasculature of the tissue, and obtaining an image of invisible light wavelength emissions, wherein the image is a representation of the vasculature of the tissue, and wherein the vasculature of the tissue indicates the desirability of the flap design, the viability of the flap, or the suitability of a failing flap for salvage therapy with fibrinolytics.

An “invisible light fluorophore” (ILF) is a compound that emits light in the invisible light region of the spectrum (670 nm to 100,000 nm), such as near infrared (670 nm to 1000 nm) to mid infrared (1000 nm to 20,000 nm) to far infrared (20,000 nm to 100,000 nm), as any light above 670 nm is invisible to the naked human eye. These invisible light fluorophores do not substantially change the appearance of the surgical field, and because tissue autofluorescence at these wavelengths is generally low, detection is extremely sensitive. Hence, invisible light fluorophores are ideal reagents for surgical imaging. In some embodiments, ILFs can also include fluorophores that are visible to the naked human eye, as long as they also fluoresce in the invisible light region.

The term “near infrared fluorophore” refers to compounds that fluoresce in the near infrared region of the spectrum, i.e., about 680 nm to 1000 nm. These substances include indocyanine green (ICG), IRDye™78 (LI-COR, Lincoln, Nebr.), IRDye80, IRDye38, IRDye40, IRDye41, IRDye700, IRDye™800CW (LI-COR; referred to below as CW800 or CW800-CA, i.e., the carboxylic acid form of the NIR fluorophore IRDye™800), Cy5.5, Cy7, Cy7.5, IR-786, DRAQ5NO (an N-oxide modified anthraquinone), quantum dots, and analogs thereof, e.g., hydrophilic analogs, e.g., sulphonated analogs thereof. In some embodiments, the NIRF is a fluorophore described in U.S. Provisional Patent Application No. 60/835,344.

The present methods have a number of advantages. For example, the dyes used herein can generally be used at low concentrations, yet are still able to provide excellent visualization. For example, the doses of methylene blue (MB) used in the fluorescence imaging methods described herein are significantly lower than those needed when MB is used as a visual (blue) dye, thus avoiding the significant toxicity issues associated with MB. Surprisingly, even at these low doses, MB is secreted or partition preferentially into urine and bile at concentrations that are optimal for fluorescent visualization, though such concentrations (e.g., a concentration in the tissue of only about 10-40 μM, e.g., about 20-μM) are far below those useful for visual light imaging.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1A is a line graph illustrating the change in fluorescent intensity with increasing concentrations of methylene blue (MB) in phosphate buffered saline (PBS, filled squares), 100% fetal bovine serum (FBS, open triangles pointing up, dashed line), or methanol (MtOH, open triangles pointing down, solid line).

FIGS. 1B and 1C are bar graphs illustrating peak absorbance wavelength (1B) and peak fluorescence emission wavelength (1C) for increasing concentrations of MB in PBS (black bars), FBS (grey bars), or MtOH (white bars).

FIG. 1D is a line graph showing the fluorescence emission decrease of MB at pH=6, in acidic conditions, and with the addition of Sodium Bicarbonate. Asc, ascorbic acid.

FIG. 2 is a set of nine images of a rat heart at sequential time points (0 seconds, top row; 12 seconds (middle row); and 5 minutes (bottom row)) after iv administration of MB. Color images are shown in the left column, near infrared (NIR) images are shown in the middle column, and color/NIR overlay images (merged) are shown in the right column. To create a single image that displays both anatomy (color video) and function (IR fluorescence), the IR fluorescence image was pseudo-colored in lime green and overlaid with 100% transparency on top of the color video image of the same surgical field.

FIG. 3A is a pair of line graphs illustrating signal to background ratio (SBR) in common bile duct (CBD) versus pancreas (left graph) or liver (right graph), at sequential time points after administration of MB.

FIG. 3B is a trio of images of rat CBD imaged after iv administration of MB. Color images are shown in the left column, near infrared (NIR) images are shown in the middle column, and color/NIR overlay images (merged) are shown in the right column.

FIG. 4 is a trio of images of rat CBD imaged after direct administration of MB. Color images are shown in the left column, near infrared (NIR) images are shown in the middle column, and color/NIR overlay images (merged) are shown in the right column.

FIG. 5 is a set of nine images of CBD imaging with ICG (top row), IR-786 (middle row), and CW800CA (bottom row), which are able to provide visualization of the bile duct 10 min after injection of 50 μL of 1000 μM ICG and 50 μL of 100 μM the other two NIR fluorophore solutions into portal vein of the rats. Shown are a still from color video (left), NIR fluorescence (middle), and merged images of the two (right). CBD can be visualized by all 3 contrast agents (arrowheads), but CW800CA shows no dye stacking in the liver and a higher SBR than the other two. NIR fluorescent exposure time is 200 msec.

FIGS. 6A-D are line graphs illustrating quantification of the fluorescence emission and signal to background ratio (SBR) kinetics for CW800CA. The SBR (mean ±SEM) of the common bile duct relative to the pancreas (6A) and liver (6B) were quantified over time following intravenous injection of 50 μL of 10, 20, 50, and 100 μM CW800CA solution. The SBR to the pancreas (6C) and liver (6D) were also quantified after portal injection of 50 μL of 10, 20, 50, and 100 μM CW800CA solution.

FIGS. 7A and 7B are sets of three images of CBD mapping in a pig. The CBD was identified under normal conditions (7A) and after the insertion of 3 beads of 2.5 mm and 1 bead of 3.5 mm in diameter into the CBD from papilla vater (7B). 5 mL of a 50 μM CW800CA (7.5 μg/kg total fluorophore) was injected intravenously and imaged at 15 min. Shown are stills from color video (left), NIR fluorescence (middle), and merged images of the two (right). NIR fluorescence exposure time was 200 msec. All beads could be detected perfectly in each trial.

FIG. 7C is a trio of images of pig CBD imaged after administration of MB and ICG. Left, color image; middle, ICG NIR fluorescent image (800 nm); and right, methylene blue NIRF image (700 nm). In these images, ICG highlights blood vessels, while MB shows the CBD.

FIG. 8 is a series of three images of thoracic duct mapping in a pig. Shown are stills from color video (left), NIR fluorescence (middle), and merged images of the two (right).

FIG. 9 is a set of twelve images of thoracic duct mapping in a rat using ICG (left column), ICGHSA (second column), CW800CA (third column), and HSA800 (last column). Shown are stills from color video (top row), NIR fluorescence (bottom row), and merged images of the two (middle row).

FIGS. 10 and 11 are line graphs illustrating quantification of the fluorescence emission and signal to background ratio (SBR) kinetics for CW800CA. The SBR (mean ±SEM) of the ureter relative to the kidney (10) and abdominal wall (11) were quantified over time following intravenous injection of 50 μL of 10, 20, 50, and 100 μM CW800CA solution.

FIG. 12 is a series of three images of ureter mapping in a rat. Shown are stills from color video (left), NIR fluorescence (middle), and merged images of the two (right).

FIGS. 13 and 14 are graphs illustrating the results of HPLC/mass spectrometry metabolic analysis of CW800 carboxylic acid after excretion into urine. As shown, by the fluorescence chromatograph, the dye appears as essentially a single peak with the expected mass spectrum.

FIG. 15 is series of eight color video (left column) and near-infrared fluorescence (right column) images of skin during intravenous injection of 0.5 mg (14 μg/kg) indocyanine green (ICG) into a 35 kg Yorkshire pig. Shown are pre-injection autofluorescence (top), arterial filling at 5 sec post-injection (second row) and venous filling at 10 sec post-injection. Note that the nipple in the field (top right) serves as an additional internal control for arterial vs. venous phases.

FIG. 16 is a panel of sixteen images of methylene blue and ICG IV injection, showing CBD and Cystic Artery (CyA), as well as coronary artery, in an intact (top 3 rows) and resected (bottom row) heart.

FIG. 17 is a panel of images showing the results of parathyroid gland imaging.

### DETAILED DESCRIPTION

Intraoperative imaging is an indispensable diagnostic tool for identification of normal or abnormal anatomical structures. Surgeons usually use fluoroscopy, ultrasound, injection of high doses of a blue dye (e.g., methylene blue (MB), Lymphazurin™, or Evan's Blue), or radioscintigraphy for intraoperative imaging. These methods currently play significant roles, however, each method has some drawbacks, such as irradiation (associated with fluoroscopy and radioscintigraphy), low specificity (ultrasound), and low sensitivity (visual blue dyes). In some cases, for example MB, the doses required to achieve visual identification are associated with significant toxicity.

The methods described herein take advantage of the properties of fluorescent dyes, e.g., invisible light (IL) fluorescent dyes, to provide safe, specific, and sensitive methods for labeling and detecting anatomical structures intraoperatively. These methods include the use of dyes that are filtered and excreted by the kidney (for labeling of ureters) or taken up by the liver and secreted into bile (for labeling of the biliary tree). The preferred dyes are not significantly reabsorbed or metabolized in the body. The methods can include modifying a known dye to affect its filtration and metabolic properties; for example, pegylation of a dye can be used to significantly reduce liver uptake, thereby partitioning intravenously-injected dye into the urinary tract.

### Imaging Methods

The methods described herein can be practiced with any intraoperative imaging system that can detect invisible light (IL) fluorescence in vivo, e.g., the systems described in De Grand and Frangioni, Technol. Cancer Res. Treat. 2(6):553-62 (2003); U.S. Pat. App. Pub. No. 2006/0108509 to Frangioni et al.; U.S. Pat. App. Pub. No. 2005/0285038 to Frangioni; U.S. Pat. App. Pub. No. 2005/0020923 to Frangioni et al.; and U.S. Pat. App. Pub. No. 2005/0182321 to Frangioni, all of which are incorporated herein by reference.

The methods described herein can be used as part of an imaging system, e.g., a planar or tomographic imaging system, for high sensitivity detection of fluorescent events, thus, the methods are ideal for intraoperative imaging. Moreover, the methods described herein can be used to provide color, fluorescence, and merged images simultaneously, which allows surgeons to keep track of the fluorescence over the surgical field in real time as surgical procedures are ongoing. Depending on the strength of the fluorescence, and the location and size of the structure desired to be imaged, fluorescence that is up to several millimeters from the surface can be detected with planar reflectance imaging. Deeper tissues can be imaged using tomographic imaging methods, such as frequency-domain photon migration or time-domain techniques, which will likely extend depth detection to the 4- to 10-cm range (reviewed in Sevick-Muraca et al., Curr. Opin. Chem. Biol., 6:642-650 (2002) and Ntziachristos et al., Eur. Radiol., 13:195-208 (2003).

To highlight selected anatomical structures, two main broad strategies can be used for introduction of the dyes described herein:

1) Intravenous injection. This method requires that the dye pass through the organ into the bodily fluid, e.g., circulate in the bloodstream, be taken up by the liver or kidney, and secreted into bile or urine; and

2) Direct injection into the structure to be visualized. Direct injection also has two possibilities:
i) Anterograde injection, e.g., injection at the “top” of the structure, wherein the dye follows the normal flow of the bodily fluid, e.g., after injection into the left or right hepatic duct, the dye flows distally to visualize CBD; and
ii) Retrograde injection, e.g., cannulation of the distal orifice of a structure, which involves injecting the dye against the natural stream of the fluid.

Ureter Imaging

The ureters connect the kidney to the bladder, carrying urine laden with toxins absorbed from the bloodstream by the kidneys, leakage of which into the body cavity can cause peritonitis. During abdominal surgeries such as caesarian sections and urological surgeries, it is crucial that the surgeon be able to identify the ureters, e.g., to avoid damaging them, or to repair them after iatrogenic or external damage.

Methods for imaging the ureters include injecting an invisible light fluorophore (ILF) into the bloodstream, or direct cannulation, either anterograde or retrograde, into the ureters or bladder, such that it appears in the urine stream.

To image the ureters, it is desirable to use an IL fluorescent compound that is filtered by the kidney into the urine. Such agents will generally have a hydrodynamic diameter of less than 5 nm; are hydrophilic; and are not significantly positively charged. Agents that can be used include NIR fluorophores such as methylene blue, IR-786, CW800-CA, Cy5.5, Cy7, Cy7.5, IRdye™800CW (LICOR), and IRdye78 (LICOR).

The level of hydrophilicity of a compound plays a role in directing uptake to the kidney and/or liver; therefore, the hydrophilicity of a modifiable agent can be increased, e.g., by increasing the level of sulphonation, to increase uptake by liver and/or kidney. As described herein, agents that are unsulphonated, monosulphonated, or disulphonated are generally rapidly sequestered by the liver, but are not secreted into bile efficiently, and are thus not particularly useful to image the biliary tree. Agents that are trisulphonated, tetrasulphonated, pentasulphonated, or heptasulphonated are more likely to be secreted by the liver into bile, and are also more likely to be available in the circulation for filtration by the kidney into urine, and are thus useful for imaging the ureters and biliary tree. Agents that can be modified in this way include cyanine dyes such as Cy5.5 (Amersham Biosciences), e.g., by sulphonation.

Some agents are naturally taken up by the liver when injected systemically. To selectively label the ureters, such agents should be modified, e.g., by addition of a moiety such as a PEG i.e., by pegylation, that prevents uptake by the liver to improve their specificity for the ureters. Methods for pegylating compounds are known in the art.

Biliary Tree Imaging

Intraoperative cholangiography (IOC) has been shown to decrease the risk of common bile duct injury during invasive surgical procedures such as cholecystectomy. Invisible light (IL) fluorescence bile duct imaging offers several advantages over standard IOC with x-rays including no ionizing radiation, real-time imaging, and simultaneous imaging with the color video image of the surgical field.

In the methods described herein, ILFs that are filtered by the liver into the bile stream allow visualization of structures of the biliary tree, including the common bile duct (CBD), which is vulnerable to accidental injury during surgery as it is hidden by, and difficult to distinguish from, other tissues.

Methods for imaging the biliary tree include injecting an ILF into the bloodstream such that it appears in the bile stream. In general, those agents that will label the ureters will also label the biliary tree; therefore, agents that have properties similar to those described above for labeling ureters can be used. ILFs that can be used include NIR fluorophores such as methylene blue, indocyanine green (ICG), IR-786, and CW800-CA, Cy5.5, Cy7, Cy7.5, and IRDye™78.

For rapid and selective imaging of the biliary tree, an ILF can be injected directly into the portal vein. Direct cannulation into the right or left hepatic duct for anterograde or retrograde labeling of the biliary tree including the CBD can also be used.

Thoracic Duct

The thoracic duct is an important part of the lymphatic system, collecting most of the lymph in the body and draining it into the systemic circulation. Thus, methods for imaging the thoracic duct include injecting an IL fluorescent compound, e.g., a NIR fluorescent compound, into a lymph node, e.g., the inguinal lymph node, and detecting a fluorescence signal from the thoracic duct.

In some embodiments, the methods described herein for imaging the thoracic duct include the use of methylene blue as the ILF.

Vasculature

The ability to clearly identify vasculature in real-time during surgical procedures such as cardiac surgery, neurosurgery, and general surgery would be invaluable to the surgeon. As described herein, it has been discovered that ILFs, including methylene blue can be used for real-time IR angiography of blood vessels, e.g., to allow assessment of vessel patency, with a good signal-to-background ratio.

Tissue Flaps for Reconstructive Surgery

In general, reconstructive surgery is based on principles of tissue movement and tissue auto-transplantation. Most complex reconstructive procedures make use of “flaps,” which are blocks of tissue that can be moved or transplanted based on the anatomic characteristics of the blood supply to the flap. Such flaps may be composed of skin, muscle, bone, tendon, nerve, blood vessel, intestine, and various combinations of these types of tissue. These flaps can be moved from a “donor” site, when such donation has acceptable morbidity, to the “recipient” site, where reconstruction is needed. For example, a composite free flap of fibula and overlying skin can be used to reconstruct a mandible and floor-of-mouth defect following a major oral cancer resection. Other examples include neuromuscular flaps for facial nerve paralysis, fibular osteocutaneous flaps for mandible and oral reconstruction, breast reconstruction following breast cancer, lower extremity salvage following trauma, and total penile reconstruction.

The single most important, defining, and clinically significant aspect of a flap is its vascular supply. Obviously, if the vascular supply to a flap is not adequate, the flap will become ischemic and fail when transferred to its new position. The vascular supply to a block of tissue, known as a “pedicle,” must be adequate to supply the planned flap. The pedicle must be either maintained during the surgery (pedicled flap), or if it must be divided and freed completely from the body to reach the recipient site, it must be reanastomosed to blood vessels in the recipient area using microsurgical technique and microsurgical instrumentation (free flap).

The peripheral blood supply to the skin in most free flap donor sites arises from one of two sources: septocutaneous vessels, which course between muscle groups, and perforator vessels, which penetrate muscles underlying the skin and emanate directly from the muscle in discrete, but variable, sites. The inconsistent anatomy of the small arterial perforators within free tissue transfers that are vital to the survival of the key cutaneous portions of the flap has been a major roadblock in reconstructive surgery.

The methods described herein provide not only for the identification and location of perforators, but also their relative size and direction of arborization, permitting substantial design improvements when they are needed, prior to making an incision. After flap elevation, the methods can be used to confirm that the key perforators were not injured during the dissection and that the vascular supply to the flap is adequate. In addition, the methods provide an objective means of determining areas of flaps destined for necrosis, so that flap modification or alternative corrective measures could be taken intraoperatively. Finally, the imaging methods described herein are useful in postoperative monitoring of the flap, as flaps must be continuously monitored in the early days following free tissue transfer for evidence of arterial of venous thrombosis, since there is only a brief interval during which emergency reoperation for flap salvage is possible. The methods can also be used for assessing flap viability and guiding surgical efforts at flap salvage, including the use of fibrinolytic agents, when complications occur.

Heart Myocardium

During cardiac artery bypass grafting (CABG) and other cardiac surgery, it is important to assess myocardial perfusion. A NIR fluorescent tracer that accumulates in the heart after intravenous injection, such as methylene blue, would permit non-invasive imaging of cardiac perfusion.

Parathyroid Glands

The ability to locate the parathyroid glands during neck surgery can be a crucial determinant of success, whether the parathyroid glands are the intended surgical target or are to avoided during a procedure. In some embodiments, the methods can be used to detect, e.g., parathyroid adenomas. See, e.g., Keaveny et al., Brit. J. Surg., 1969; 56(8):595-597.

Tumors and Pancreas/Insulinoma

Methylene blue has previously been demonstrated to be useful as a visual dye of the pancreas and insulinomas. See, e.g., Keaveny et al., Brit. J. Surg., 1971; 58(3):233-234. The methods described herein can be used to identify the pancreas, insulinomas, and other tumors due to the tendency of MB to collect in those tissues; the MB can be administered systemically or directly to the organ, e.g., into the vasculature of the organ (e.g., by cannulation). Systemic administration of the dye can also be used to identify metastatic tumors from tissues that have high uptake of MB, e.g., tumors of pancreatic, e.g., insulinoma, origin.

### Fluorescent Dyes

Any IL fluorescent compound that is detectable in the tissue to be imaged, e.g., the ureter, thoracic duct, biliary tree, or vasculature, can be used. In general, those compounds that fluoresce in the NIR range (670-1000 nm) are useful. In addition, agents for use in imaging the ureters should not be significantly reabsorbed or metabolized by the body, e.g., by the liver or kidneys. A number of compounds that we have found are suitable for imaging the ureters are known in the art, including organic fluorophores, the most common of which are polymethines. One important class of these molecules is the heptamethine cyanines, comprised of benzoxazole, benzothiazole, indolyl, 2-quinoline, and 4-quinoline subclasses. Exemplary compounds include methylene blue (MB), indocyanine green (ICG), IR-786, and CW800-CA.

In some embodiments, the near-infrared fluorophore has a structure of Formula I:

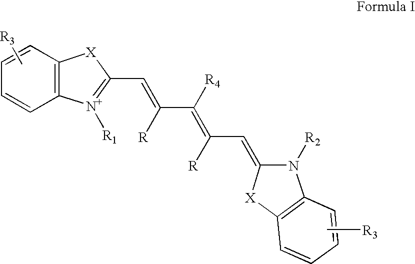

wherein, as valence and stability permit,

X represents C(R)₂, S, Se, O, or NR₅;

R represents H or lower alkyl, or two occurrences of R, taken together, form a ring together with the carbon atoms through which they are connected;

R₁ and R₂ represent, independently, substituted or unsubstituted lower alkyl, lower alkenyl, cycloalkyl, cycloalkylalkyl, aryl, or aralkyl, optionally substituted by sulfate, phosphate, sulphonate, phosphonate, halogen, hydroxyl, amino, cyano, nitro, carboxylic acid, or amide, or a pharmaceutically acceptable salt thereof;

R₃ represents, independently for each occurrence, one or more substituents to the ring to which it is attached, such as a fused ring, sulfate, phosphate, sulphonate, phosphonate, halogen, lower alkyl, hydroxyl, amino, cyano, nitro, carboxylic acid, or amide, or a pharmaceutically acceptable salt thereof;

R₄ represents H, halogen, or a substituted or unsubstituted ether or thioether of phenol or thiophenol;

R₅ represents, independently for each occurrence, substituted or unsubstituted lower alkyl, cycloalkyl, cycloalkylalkyl, aryl, or aralkyl, optionally substituted by sulfate, phosphate, sulphonate, phosphonate, halogen, hydroxyl, amino, cyano, nitro, carboxylic acid, amide; or a pharmaceutically acceptable salt thereof.

In some embodiments, the two occurrences of R taken together form a six-membered ring. In some embodiments, R1, R2, and one or both R3 include sulphonate.

Methylene Blue (MB)

Although it was previously known to use MB as a visual dye, the use of MB in fluorescence imaging has not been significantly appreciated. As described herein, methylene blue (MB) has fluorescent properties. The emission wavelength (670 nm to 720 nm with a peak that shifts as a function of dye concentration) is within the NIR range at physiologically safe concentrations and therefore permits high sensitivity and high signal to background due to low autofluorescence in humans and animals. This characteristic allows MB to be used as a vascular contrast agent, using fluorescence imaging technology. Surprisingly, MB is secreted or partitions specifically into certain fluids and organs, including the thoracic duct, bile (allowing visualization of biliary tree), urine (allowing visualization of the ureters), heart myocardium, vasculature (allowing imaging of, inter alia, the myocardium, coronary artery, etc.), and pancreas (e.g., into beta cells, allowing visualization of that organ and tumors and metastases with a pancreatic origin, e.g., insulinomas).

MB has the advantage of already being approved by the U.S. Food & Drug Administration as a blue dye to assess gastrointestinal tube placement and as a treatment for methemoglobinemia. However, thoracic ducts can be visualized clearly only after direct injection of high doses into the inguinal lymph node in pigs using simple naked-eye color detection. But as demonstrated herein, the thoracic ducts can be detected using MB fluorescence at much lower concentrations, even using the same injection technique. For example, when used as a blue dye to map thoracic duct, MB is injected directly into the thoracic duct as a 1% (31 mM) solution in sterile pH-adjusted water. When used as a fluorophore, and injected directly into the thoracic duct, one needs to achieve a concentration in the tissue of only about 10-40 μM, e.g., about 20-30 μM (which is about a 1,000 fold lower dose). If injecting a distal node that would drain into the TD, a concentration between 30 μM and 31 mM can be used. This suggests that fluorescence imaging is more sensitive than visual inspection with human eyes or color cameras.

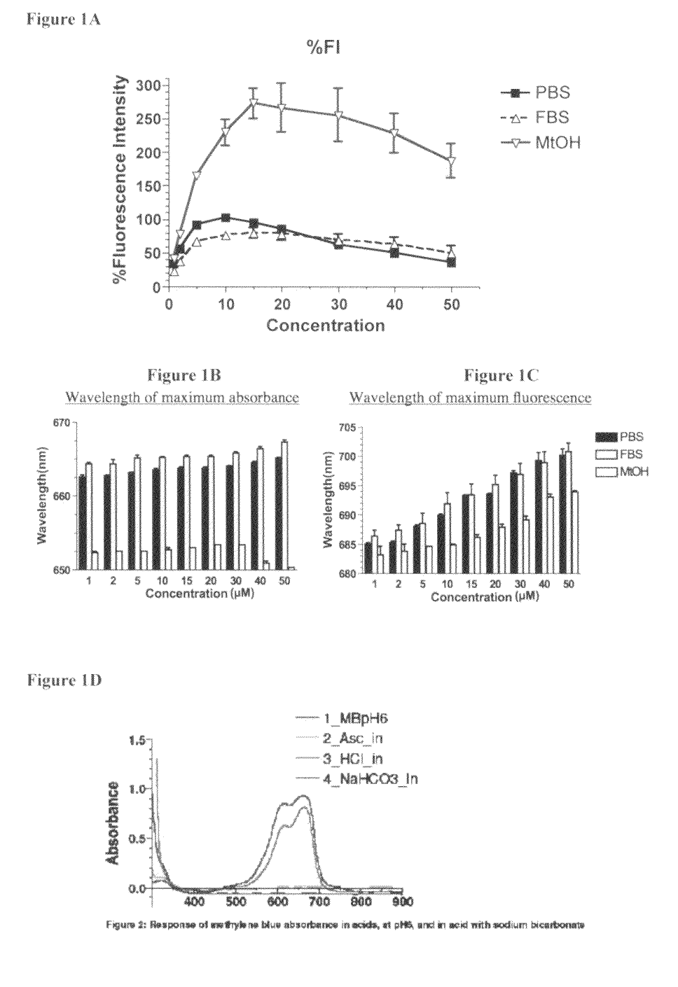

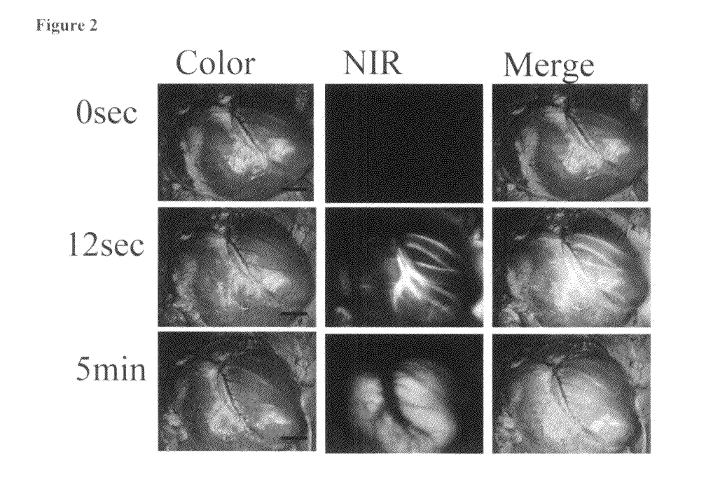

Doses of 1.0-2.0 mg/kg of methylene blue are widely used clinically for the treatment of methemoglobinaemia, and much larger doses (on the order of 4.0-7.5 mg/kg) are administered for parathyroidal adenoma/hyperplasia detection. At the higher end, e.g., 7.5 mg/kg, MB administration sometimes causes severe adverse reactions, e.g., methemoglobinaemia or anaphylaxis. In addition, there are some reports indicating that intradermal injection of MB can cause skin damage. For example, the high doses used for sentinel node detection, e.g., around 4 ml of 30 mM MB, are associated with reports of injection site reactions. At these high concentrations, no fluorescence would be visible due to the concentration-dependent quenching of MB emissions. Thus, in general, the doses used in the methods described herein are about 10 times lower, and in some embodiments 100 times lower than those previously used, and are expected not to cause either skin damage or adverse reactions. For example, in some embodiments, the methods include the administration of a solution including at least 0.03% MB, e.g., about 0.03 to 10% MB, e.g., 0.05% to 10%, e.g., 1% to 3.5%. These percentages are weight/weight, i.e., a 10% solution is 100 mg/ml. In general, the total dose that will be used for most applications is about 1-4 mg/kg of body weight when administered systemically. So, for a 70 kg human, and a desired systemic dose of about 1 mg/kg, one would need 70 mg, which is equal to 7 ml of a 10%=100 mg/ml solution or 70 ml of a 1%=10 mg/ml solution. It is desirable to achieve a concentration in the tissue to be imaged of about 10-40 μM, e.g., about 20-30 μM. The concentration can vary depending on the local environment of the structure to be imaged, e.g., the pH of the environment, or the concentration of proteins. In some embodiments, an optimal concentration can be identified based, e.g., on the graphs in FIGS. 1A-D.

In summary, the MB fluorescence imaging methods described herein realize higher sensitivity with lower doses of MB. Methylene blue can be used as a lymphatic tracer, a bile duct and ureter indicator, and a vascular contrast agent. These broad indications introduce more options for intraoperative imaging. In addition, methylene blue can be used in combination with other fluorescent agents, such as ICG, to provide multi-wavelength, multi-color fluorescence imaging.

NIR Fluorophores: ICG, MR-786, and CW800-CA

ICG is a di-sulphonated heptamethine indocyanine that is FDA-approved for cardiac and hepatic function studies. CW800-CA is a carboxylic acid analog of IRDye™800CW, a newer heptamethine indocyanine with higher quantum yields and molar extinction coefficients. IR-786 is a heptamethine indocyanine with no sulphonation, and is an extremely hydrophobic agent. On the other hand, CW800-CA is a tetra-sulphonated heptamethine indocyanine, which increases its hydrophilicity.
CW800-CA (LI-COR Inc.): The carboxylic acid of IRDye™800-CW prepared from the commercially available N-hydroxysuccinimide ester, by hydrolysis of the ester in water at pH 8.5. This is a tetra-sulphonated heptamethine indocyanine with emission ≈800 nm. After intravenous injection it is rapidly cleared by: 1) the liver and excreted into bile and 2) the kidneys and excreted into urine. Thus, this dye is useful for imaging the biliary tree and ureters.
ICG (Akorn, Inc.): Commercially available and FDA-approved near-infrared heptamethine indocyanine fluorophore that is di-sulphonated. After intravenous injection, it is rapidly cleared from the blood by the liver, but is only inefficiently transported into bile. ICG can be used to image the structures described herein when administered by direct injection or cannulation of the structure.
IR-786 (Sigma-Aldrich, Inc.): Commercially available non-sulphonated near-infrared heptamethine indocyanine fluorophore. After intravenous injection, it is rapidly extracted into many tissues in the body, especially the liver, and is inefficiently transported into bile. IR-786 can be used to image the structures described herein when administered by direct injection or cannulation of the structure.
IRDye78: Commercially available tetra-sulfonated heptamethine indocyanine-type NIR fluorophore with peak absorption at 772 nm and peak emission at 790 nm. IRDye78 can be used to image the structures described herein when administered by direct injection or cannulation of the structure. See, e.g., Zaheer et al., Mol. Imaging, 2002; 1(4):354-64.
   ICG and IR-786 are taken up almost exclusively by the liver and secreted only inefficiently into the bile, but CW800-CA is readily taken up by both kidney and liver, and exported into bile and urine. Since the major chemical difference among these molecules is the degree of sulphonation, and based on the results described herein, it is reasonable to expect that any near-infrared fluorophore will work as well or better than CW800-CA, provided that it is filtered by the kidney and/or liver. For example, suitable fluorophores can be sulphonated. Based on the results described herein, it is clear that tetra-sulphonated derivatives can be used. Furthermore, based on the results described herein, it is reasonable to expect that penta- and hepta-sulphonated derivatives should also work, and should have a more rapid onset, since their excretion into bile and urine is expected to be more rapid than the tetra-sulphonates. The tri-sulphonates will perform in an intermediate fashion with respect to di- and tetra-sulphonates.

Co-Administration

In the preclinical studies described herein, MB has been found to be useful in imaging the bile-duct, ureters and parathyroid gland. In some cases, less than optimal performance was observed in the ureter. This is because, like many other dyes, the fluorescence properties of methylene blue are sensitive to the pH of its environment. In acidic conditions the absorption and quantum efficiency of the fluorophore decreases substantially. Thus, in some cases, active buffering of the urinary pH with a urinary alkalizer (i.e., an agent that raises the pH of the urine, e.g., sodium bicarbonate or acetazolamide) is co-administered with a systemically administered dye used for imaging the ureters (e.g., with methylene blue).

In some embodiments, the methods described herein include the co-administration of one of more other compounds that enhance the performance of the dyes. For example, in some embodiments, a

### EXAMPLES

The invention is further described in the following examples, which do not limit the scope of the invention described in the claims.

### Example 1

### Optical Properties of NIR Fluorophores

This Example describes experiments that evaluated the NIR fluorescent properties of several dyes, including methylene blue (MB), ICG, IR-786, and CW800 CA, using absorbance/fluorescence spectrometry and fluorescence quantum yield measurements.

Absorbance spectrometry was performed with a 1-cm path length quartz cuvette in a USB2000 fiber-optic spectrometer (Ocean Optics, Dunedin, Fla.). Fluorescence spectrometry was performed with a 1-cm path length, 1.5 mL disposable methacrylate cuvette (Fisher Scientific, Hampton, N.H.) in a HR2000 fiber-optic spectrometer, and CUV-ALL-UV four-way cuvette holder (Ocean Optics), using a 655 nm laser diode light source (OPCOM O.E. Inc., Xiamen, China) with maximum output less than 5 mW. Data acquisition was performed with a computer using an OOI-Base32 spectrometer operating software package (Ocean Optics). The spectral range of the USB2000-FL spectrometer was from 350 to 1000 nm with spectral resolution of 7.6 nm, and that of the HR2000 spectrometer was from 200 to 1100 nm with a spectral resolution of 6.7 nm.

Fluorescence intensity was measured in each solvent (PBS, FBS, and methanol) at each concentration of the dye to determine the effect of the methylene blue concentration and solvents on total fluorescence yield. Fluorescence quantum yields of methylene blue in PBS, FBS, and methanol were calculated using oxazine725 in ethylene glycol (quantum yield: 19%) as a calibration standard, under the conditions of matched absorbance of methylene blue in each solvent.

Methylene Blue (MB)

MB is a small molecule (M.W. 320 Da) cationic thiazine dye that is FDA-approved for diagnostic agent and indicator dye in humans. In this example, MB purchased from Mayne Pharma Inc. (Paramus, N.J.) was diluted in adequate amount of phosphate-buffered saline (PBS) to obtain 1, 2, 5, 10, 15, 20, 30, 40, and 50 μM before use.

The optical properties of MB are summarized in Table 1; the molar extinction coefficient in FBS is 76.000, and fluorescence quantum yield in FBS is 2.5%. These results indicated that ICG fluorescence, which has an extinction coefficient and fluorescence quantum yield in FBS of 168,000 and 9%, respectively, is about 8 times larger than methylene blue fluorescence per se.

TABLE 1
Physical and Optic Properties of Methylene Blue and ICG

| | | Molar | Excitation | Emission | | | |
|---|---|---|---|---|---|---|---|
| | | Extinction | Wavelength | Wavelength | Fluorescence | | |
| | | Coefficient | Maximum | Maximum | Quantum | Excretion | Excretion |
| | | (M⁻¹cm⁻¹) | (nm) | (nm) | Yield (%) | to | to |
| | M.W. | PBS (FBS) | PBS (FBS) | PBS (FBS) | PBS (FBS) | Bile | Urine |
| Name | (Da) | [MtOH] | [MtOH] | [MtOH] | [MtOH] | (+/−) | (+/−) |
| Methylene | 320 | 71500 (86500) | 665 (666) | 685 (685) | 5.5 (2.5) | + | + |
| Blue | | [71500] | [653] | [681] | [9.5] | | |
| ICG | 776 | 11,7000 (168,000) | 779 (800) | 806 (811) | 1 (9) | + | − |

In addition, MB fluorescence is within the NIR range, and has about a hundred nanometer difference in wavelength from ICG and indocyanine derivatives. This difference should permit the separation of these fluorescence with our fluorescence imaging system.

Environment- and Concentration-Dependence of MB Fluorescence

Methylene blue is in equilibrium with leucomethylene blue, the latter being clearn, colorless, and non-fluorescent. The conversion between the two is redox and pH-dependent, with high pH (≧6) favoring the fluorescent methylene blue form and lower pH favoring the clear, colorless, and non-fluorescent form.

Methylene blue exhibits complex fluorescence output, with peak fluorescence intensity occurring at intermediate concentrations of 4-50 μM. Intensity is also highly dependent on chemical environment, with higher output in lipid-rich environments. For the experiment shown in FIG. 1A, pure methylene blue powder was dissolved in phosphate-buffered saline (pH 7.4), 100% fetal bovine serum, or absolute methanol, at the concentration shown, and fluorescence intensity (arbitrary values) measured.

Methylene blue exhibits complex optical behavior. Its wavelength of peak extinction coefficient (i.e., absorbance) is dependent on concentration and chemical environment (see FIG. 1B), as is its wavelength of peak fluorescence emission (FIG. 1C). For this experiment, pure methylene blue powder was dissolved in PBS, FBS, or methanol, at the concentrations shown in FIGS. 1A-C, the spectrum of its absorbance and fluorescence emission obtained, and the peak of each identified.

The total fluorescence yield of methylene blue is maintained at the maximal level (1A) and peak absorbance wavelength remains steady (1B) in a relatively wide range of the dye concentration, and peak fluorescence emission wavelength does not decrease at higher concentrations (1C), allowing for adjustment of the dose of MB for imaging to optimize signal to background ratio.

Finally, the pH dependence of MB was examined, by dissolving MB in PBS (pH 6), Ascorbic Acid, HCl, and a NaHCO3-buffered solution. As shown in FIG. 1D, there was essentially no fluorescence in the acidic solutions, and significant fluorescence at the more basic pH. This is an important consideration because intermolecular quenching and internal absorption often limit the usable dose range of fluorescent contrast agents. Thus the conditions and concentration can be optimized.

NIR Fluorophores: ICG, IR-786, and CW800-CA

IR-786 and sodium ICG (Cardiogreen™) were purchased from Sigma (St. Louis, Mo.). IR786 was diluted to 100 μM in phosphate-buffered saline (PBS) supplemented with 10% Cremophor™ EL (Sigma), a polyoxyethylated castor-oil derivative, and 10% absolute ethanol.

CW800-CA is the carboxylic acid form of IRdye™ 800CW NIR dye, which was obtained as an NHS-ester from LI-COR (Lincoln, Nebr.) and hydrolyzed in a water-based buffer, pH 8.5 for one hour at room temperature before purification by HPLC on a C18 column.

10 mM stock solutions of ICG and 18 mM CW800-CA were stored in DMSO at −80° C. in the dark. ICG and CW800-CA were diluted in PBS before use.

The optical properties of these contrast agents are summarized in Table 2. All three fluorophores have similar peak absorbance and emission wavelengths, and the quantum yield of CW800-CA was the highest of the three.

TABLE 2
Physical and Optic Properties of NIR Fluorophores

| | | | Peak | | Quantum | | Excretion |
|---|---|---|---|---|---|---|---|
| | | | Absorbance | Peak | Yield | Excretion | to |
| | | Level of | in PBS | Fluorescence | in PBS | to | Urine |
| Name | M.W. | Sulphonation | (nm) | in PBS (nm) | (%) | Bile (+/−) | (+/−) |
| CW800CA | 1047 | 4 | 775 | 796 | 9 | + | + |
| IR-786 | 584 | 0 | 768 | 803 | 3.3 | + | − |
| ICG | 776 | 2 | 779 | 806 | 1 | + | − |

### Example 2

### Angiographic and Myocardial Perfusion Imaging Methods

Male Sprague-Dawley, 300 g rats obtained from Charles River Laboratories (Wilmington, Mass.) were anesthetized with 65 mg/kg IP pentobarbital. 30 kg adult female Yorkshire pigs (E.M. Parsons & Sons, Hadley, Mass.) were induced with 4.4 mg/kg intramuscular Terazol (Fort Dodge Labs, Fort Dodge, Iowa). Pigs were intubated and maintained with 1.5-2% isoflurane.

In anesthetized rats, 0.1, 0.5 and 1 mg/kg MB were injected intravenously, and then images were taken during 15 min after injection. Signal to background ratio (SBR) (the signal of left anterior descending branch of coronary artery (LAD) relative to that of chest wall, and the signal of myocardium relative to that of chest wall) was quantified using the same region of interest (ROI) through entire observation. In anesthetized pigs, 100 μL/kg of 0.5% methylene blue (1.6 μmol/kg) was injected intravenously and then the hearts were observed using the imaging system described herein. For multi-wavelength fluorescence imaging, 0.25 ml of 100 μM ICG (75 nmol/kg) was injected intravenously in rats and 0.06 mg/kg ICG (75 nmol/kg) in pigs.

The near-infrared imaging system used in this and the following examples has been described previously in detail (see, e.g., De Grand and Frangioni, Technol. Cancer Res. Treat. 2(6):553-62 (2003); U.S. Pat. App. Pub. No. 2006/0108509 to Frangioni et al.; U.S. Pat. App. Pub. No. 2005/0285038 to Frangioni; U.S. Pat. App. Pub. No. 2005/0020923 to Frangioni et al.; and U.S. Pat. App. Pub. No. 2005/0182321 to Frangioni). The system was adjusted to account for the optical properties of methylene blue. Briefly, the light source of the original system is composed of a white light source and a near-infrared light source. The surgical field is illuminated with both lights, and its excitation fluence rate is 5 mW/cm². White light and near-infrared images were able to be captured separately using a dichroic filter cube set, and these images could be merged. Images were captured and refreshed 15 times a second. This capability, with zooming and auto-focus function, and a cardiac gating function (which provides a clear image of the heart with few motion artifacts by synchronizing the timing of image capture with cardiac contraction, and displays three images at the same time), allows real-time, precise identification of the anatomical structure.

The system was modified in 3 ways for use with MB. First, the white lights were filtered with HQ675SP (Chroma Technology, Brattleboro, Vt.). Second, 4 pairs of 400 mA, 660 nm LEDs were added and filtered with HQ650/45X (chroma.) instead of the near-infrared light source. Excitation fluence rate with this new light source is 1 mW/cm². Third, the dichroic filter and emission filter were changed to 680 dcxr (chroma.), HQ700/35 (chroma.), respectively. This allows effective separation of white light and fluorescent light coming from methylene blue, and provides color and fluorescence images separately, and allows display of the two images with a merged image simultaneously in real-time.

Angiographic and Myocardial Perfusion Imaging Using MB

Autofluorescence out of normally perfused rat hearts was minimal.

MB was used to visualize coronary arteries, but peripheral branches and coronary vein were not detectable using MB in rats. Intravascular (arterial) signals were not detected with lower doses of methylene blue (0.1 mg/kg); with larger amounts (0.5 and 1.0 mg/kg), coronary arteries could be detected. Methylene blue is rapidly distributed from the bloodstream to myocardial tissue including the myocardium, which likely explains why the coronary venous return cannot be visualized.

A homogenous signal from the heart wall was detectable in all loading doses of MB, which suggested that MB can be used as an optical indicator of myocardial perfusion. The intensity was dependent on the loading dose of methylene blue, however, the decrease of the fluorescence in the cardiac wall was linear and the slope was constant and independent of loading dose.

In a normally perfused pig's heart, autofluorescence was minimal. Following intravenous injection of methylene blue via the external jugular vein, high signal to background (SBR) images of arterial blood flow in the beating heart were obtained (FIG. 2, top row), and subsequently, homogenous fluorescence out of the whole heart wall with a average SBR of about 1.5 (FIG. 2, middle). When acute arterial occlusion was introduced by ligation of a peripheral diagonal branch of the left anterior descending artery and methylene blue was injected intravenously, myocardium distal to the occlusion had five-fold lower signal than the well-perfused part of the myocardium, and the part looked defective on the NIR image (FIG. 2, bottom).

### Example 3

### Cholangiographic Imaging

Extrahepatic bile duct is often difficult to identify without contrast agents in rats. The use of NIR agents including methylene blue was evaluated in the experiments described in this Example. The imaging system described in Example 2 was used in these experiments as well.

Methylene Blue

In anesthetized rats, 100 μL/kg of 1% MB (1 mg/kg) was injected intravenously via penile vein. A simultaneous color video/NIR fluorescence intraoperative imaging system was employed for quantification of fluorescence images. For multi-wavelength fluorescence imaging, 0.25 ml of 100 uM ICG (75 nmol/kg) was injected intravenously in rat and hepatic arteriography and portography can be seen.

In anesthetized pigs, bile duct was visualized by intravenous injection of 100 μL/kg of 1% MB, or direct cannulation into the common bile duct for injection of 50 μM of MB. Fluorescence intensities of bile duct, liver (right lobe), and abdominal wall were measured at 3, 5, 10, 20, and 30 minutes following injection. For hepatic arteriography and portography, 0.06 mg/kg ICG (75 nmol/kg) is injected intravenously.

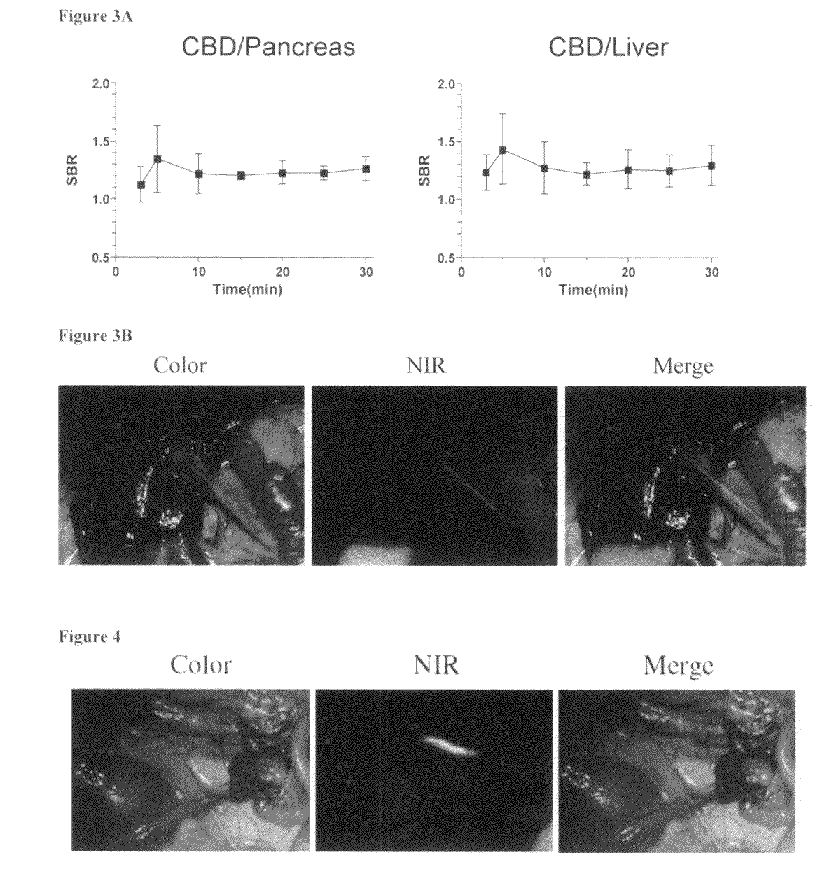

In rats, the common bile duct (CBD) was detectable just 2-3 minutes after intravenous injection of the MB. The ratio of common bile duct intensity relative to pancreas and to liver rapidly reached maximal at around 10 minutes and was steady at least 30 minutes after injection (FIG. 3A). No dye stacking in the liver was observed. The CBD could be detected only in the fluorescence image and merged image, however, not in the color image (FIG. 3B).

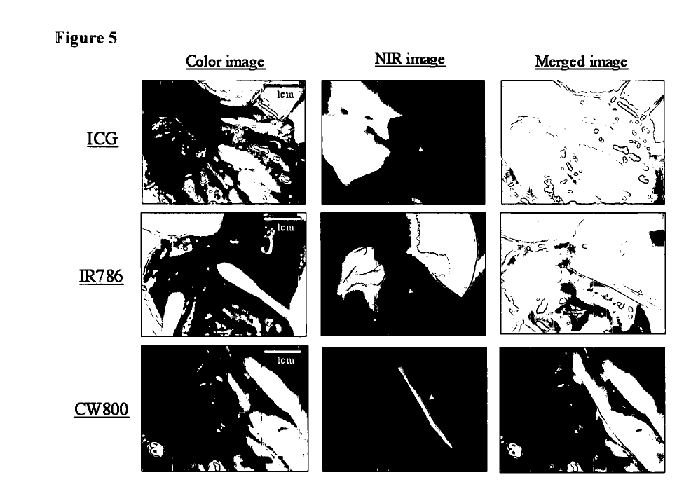

In pigs, bile had a faint fluorescence, and common bile duct was detectable as in the rat model. However, the intrahepatic bile duct was not identifiable by fluorescence during hepatic dissection. Direct retrograde administration of MB into the CBD successfully visualized the CBD (FIG. 4). This direct injection of MB into bile duct enabled easier optimization of concentration in bile than did intravenous administration. Therefore, if access to the biliary system is available, direct injection of MB is an alternative method for visualization of the biliary system.

IR-786 and CW800-CA

50 μL of 100 μM IR-786 or CW800-CA (15 μg/kg), or 1 mM ICG (150 μg/kg), was injected into portal vein in anesthetized rats. A simultaneous color video/NIR fluorescence intraoperative imaging system was employed for quantification of NIR fluorescence images. In another 32 rats, bile duct was visualized by intravenous (N=16) or portal vein injection (N=16) of 50 μL of CW800-CA at 10, 20, 50 and 100 μM to quantify the best concentration for bile duct imaging. Fluorescence intensities of bile duct, liver (right lobe), and pancreas were measured at 3, 5, 10, 20, and 30 min following injection.

Within three minutes after portal vein injection, all three contrast agents allowed imaging of the bile duct. Both ICG and IR-786 could be visualized in the CBD, but they showed significant dye stacking to the liver, which led to bright fluorescence from the liver (FIGS. 5A-B). CW800-CA also showed the CBD, with an excellent signal to background ratio (SBR), but with no significant dye stacking to the liver (FIG. 5C). Thus, CW800-CA was selected for CBD visualization.

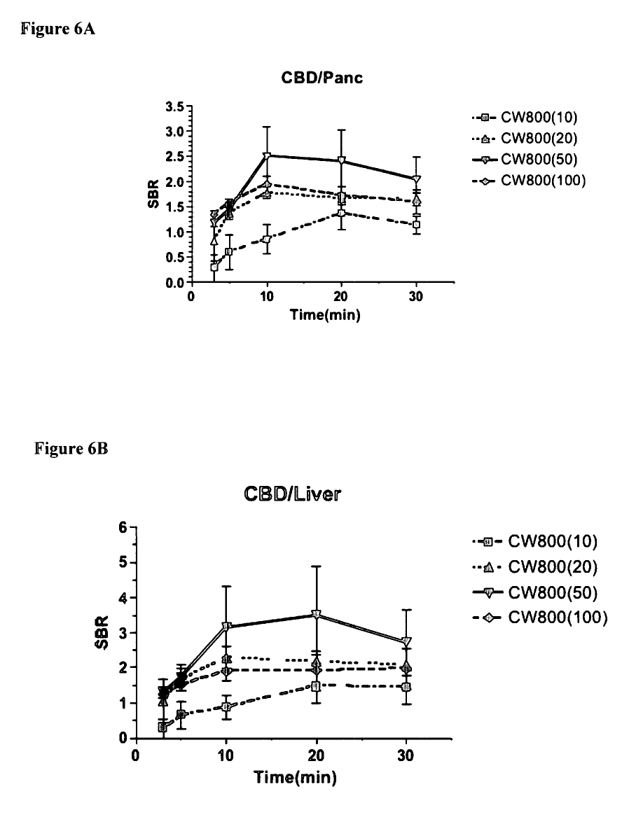

The next experiments were aimed at optimizing the dose and administrative route. As shown in FIGS. 6A-D, in general, CBD could be identified 3 minutes after either intravenous or portal injection, but portal vein injection improved the SBR, especially using 10 and 20 ∞M CW800-CA administration. Peak performance for CBD visualization appeared at approximately 10 minutes in every concentration of CW800-CA, and 50 μM CW800-CA (7.5 μg/kg) injection showed the best SBR. SBR by 100 μM CW800-CA (15 μg/kg) administration was not always better than that by 50 μM CW800-CA administration.

Based on the results obtained in rats, CW800-CA was chosen as the contrast agent for a large animal model approaching the size of human. In 7 anesthetized pigs, 5 mL of 100 μM CW800-CA (15 μg/kg total) was injected into portal vein (N=3), or 5 mL of 50 μM (N=2) or 100 μM (N=2) of CW800-CA (7.5, and 15 μg/kg total, respectively) were injected intravenously.

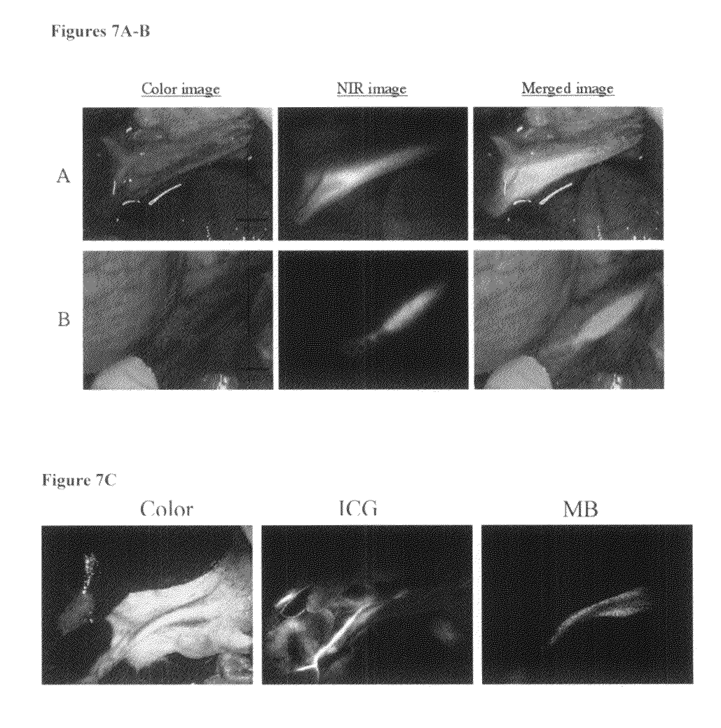

As shown in FIG. 7A, the normal CBD could be visualized using 5 mL of 100 μM CW800-CA (15 μg/kg) with both portal vein injection (N=2) and 5 mL of 50 and 100 μM CW800-CA (7.5 and 15 μg/kg, respectively) and intravenous injection (N=2, each). Visualization of the CBD was usually possible 7-10 minutes after dye injection.

Beads of 2.5 or 3.5 mm in diameter were inserted into common bile duct via papilla vateri by duodenostomy (N=2) to simulate blockage of the CBD. As shown in FIG. 7B, when the two types of beads were inserted into CBD, these beads could be detected completely in 2 of 2 pigs, which were administered 5 mL of 50 uM CW800-CA intravenously.

Multiwavelength Imaging

MB emits in the low end of the NIR range, at about 690-700 nm, and so is also ideal for use in combination with 800 nm fluorophores typically used for guidance (such as ICG) in two-channel NIR fluorescent imaging methods. Therefore, MB was used to visualize CBD in an anesthetized pig, while ICG was used to visualize vasculature.

The results, shown in FIG. 7C, demonstrate that a combination of MB with a higher wavelength emitting dye such as ICG is effective for multiwavelength imaging, allowing simultaneous identification of multiple tissue structures.

Discussion

Direct injection of blue dyes into tissues can indiscriminately change the color of tissues in the surgical field, which can lead to difficulty in dissecting tissue. Injection of blue dye directly into the common bile duct is insufficient, as the change of the bile color is not detectable to the human eye. However, in our study, CBD could be identified with NIR imaging in all rats and pigs, because NIR fluorescence from these agents within the CBD penetrate relatively deeply through the tissue, although CBD cannot be detected if the overlying tissue is too thick for the fluorescence to penetrate. In addition, autofluorescence in the range of NIR light from the tissue in the surgical field was quite low. Thus, this imaging procedure for CBD is highly sensitive and specific.

Three NIR contrast agents were compared for use in bile duct imaging: ICG, CW800-CA, and IR-786. CW800-CA proved to be the most selective, as the other two contrast agents suffered from lack of efficient secretion into bile from the liver. The other two agents have markedly hydrophobic properties, and high affinity with lipid bilayer of the cell membrane. CW800 has 4 sulphonated residues, but there is zero in IR786 and only two sulphonated residues in ICG; the number of sulphonated residues is related to the hydrophilic property of the contrast agents. In addition, CW800-CA was shown to be excreted into the bile in a substantially unmetabolized form, which increases the rapidity with which images can be obtained after administration, suggesting that it will be safe for use in humans.

The dose of CW800-CA required for CBD detection is in the range of 3-15 μg/kg for portal vein injection, and 7.5-15 μg/kg for systemic intravenous injection. The goal to dose adjustment is to get CW800-CA concentration in CBD as close to 10 μM as possible and that in the background as low as possible. Significant quenching of its fluorescence occurs above 10 μM CW800-CA (Ohnishi et al., Mol. Imaging., 2005; 4(3):172-81). It should be noted that it is unlikely that higher concentration of CW800 injection would improve the CBD imaging because of this fact.

### Example 4

### Thoracic Duct Mapping

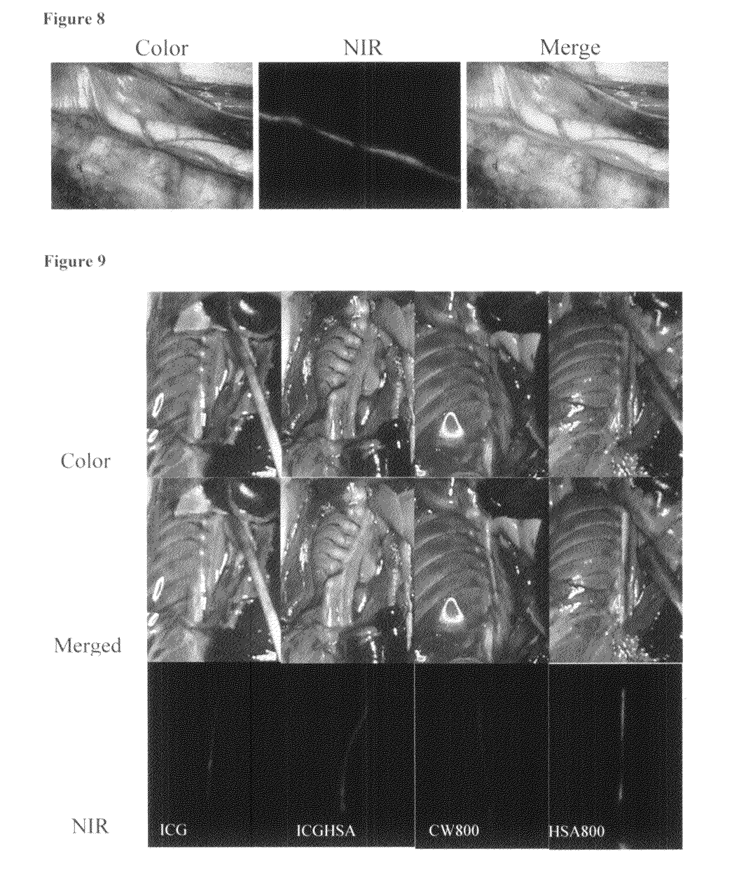

In anesthetized pigs, 5 mL of 1% methylene blue was injected into an inguinal lymph node, then the thoracic duct was observed. Thoracic duct was visualized with a very faint blue color in the color image, but was clearly visualized in the fluorescence image (FIG. 8).

Additional experiments on thoracic duct visualization in the rat model were also performed using other NIR fluorescent agents. 50 μL of a 10 μM solution of ICG, ICGHSA (a non-covalent absorption of ICG to human serum albumin), CW800CA, or HSA800 (a covalent conjugation of CWA800 to human serum albumin) was injected into mesenteric lymph node, and visualized 5 minutes later. The results are shown in FIG. 9. The thoracic duct was not visible to the naked eye in the color image, but was clearly visualized in the fluorescence image.

### Example 5

### Ureteral Mapping

In anesthetized 3 pigs, 1 mg/kg of methylene blue was injected intravenously and then the abdomen was opened surgically and ureter was identified by imaging. For pelvic arteriography, 0.06 mg/kg ICG (75 nmol/kg) was injected intravenously.

In rats, ureters were visualized by intravenous injection of 50 μL of CW800 CA at 10, 20, 50 and 100 μM to quantify the best concentration for bile duct imaging. Fluorescence intensities of ureters, kidney, and abdominal wall were measured at 3, 5, 10, 20, and 30 min following injection.

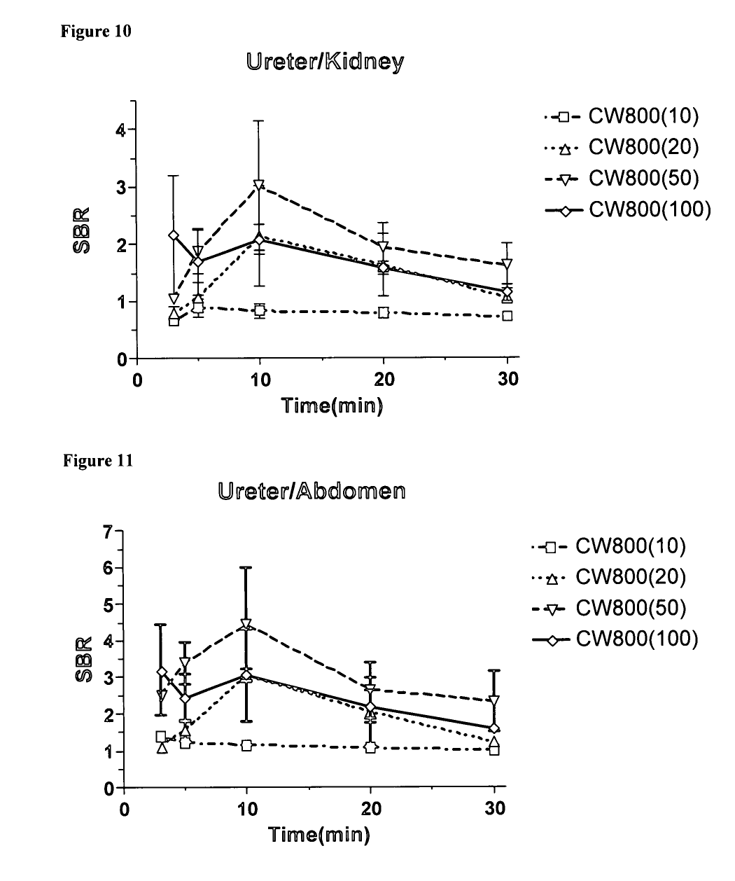

As shown in FIGS. 10-11, in general, ureters could be identified 3 minutes after intravenous injection. Peak performance for ureter visualization appeared at approximately 10 minutes in every concentration of CW800-CA, and 50 μM CW800-CA (7.5 μg/kg) injection showed the best SBR. SBR for 100 μM CW800-CA (15 μg/kg) administration was not always better than that by 50 μM CW800-CA administration.

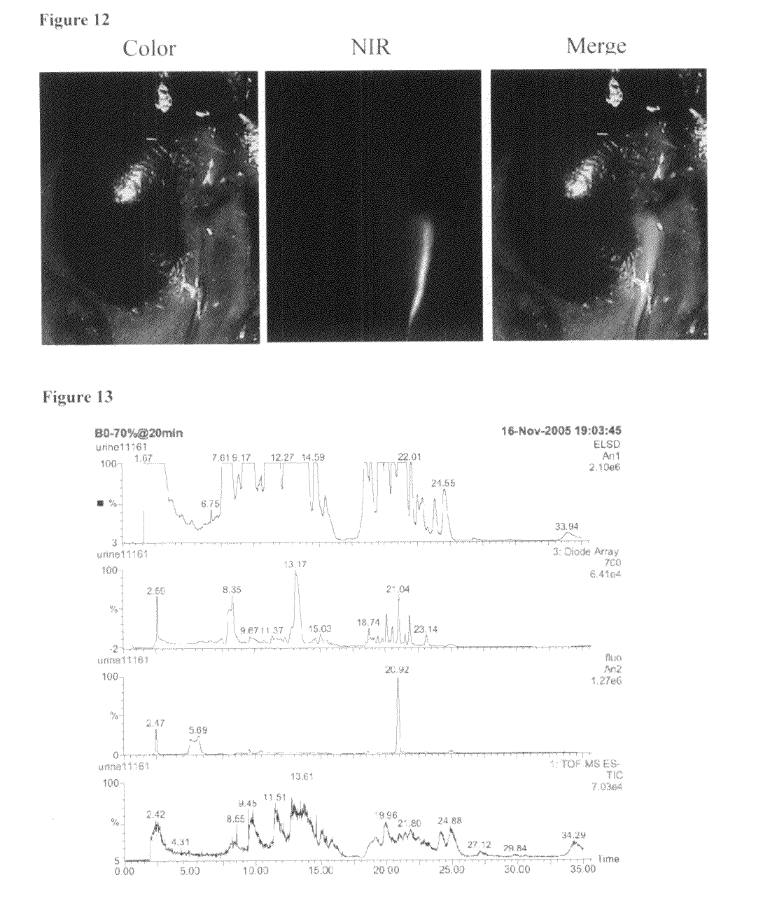

CW800CA provided excellent visualization of ureters, as is shown in FIG. 12.

### Example 6

### Metabolism of CW800-CA

As described herein, ureter visualization can usually be obtained rapidly, even in a few minutes following injection. This suggested that CW800-CA may be excreted into urine eliminated as an unchanged form, as metabolisation would likely destroy fluorescence. To clarify the point, an HPLC/mass spectrometry (using electrospray/time-of-flight (ES-TOF) method has been made.

Urine samples were collected from pigs (N=5), and analyzed. The dye usually can be pinpointed with a fluorescence detector equipped with ES-TOF mass spectrometry. Urine samples were first passed over a 6,000 Da cutoff gel-filtration column to remove contaminating proteins, and eluate less than 6,000 Da was analyzed on a C18 column. Buffer A was water, BioPlus (American Bioanalytical, Natick, Mass.) and Buffer B was acetonitrile. Using a gradient of 0 to 50% Buffer B over 30 min, urine samples were resolved on a 4.6×150 mm Symmetry C18 column (Waters) at a flow rate of 1 ml per minute with eluate fed into a Waters LCT ES-TOF mass spectrometer. Ion mode was set to electrospray negative (ES−), and cone and capillary voltages were set at 30 and 3000 (V), respectively. Desolvation and source temperature were set at 350, 140 (° C.), respectively. Data were analyzed with Masslynx (Waters) software.

Results are shown in FIGS. 13 and 14. The fluorescence from the bile sample was detected and pinpointed at the identical molecular weight of CW800-CA, which suggested that CW800-CA was excreted into bile as an unmetabolized form. The result is consistent with our expectation. This rapid elimination from the body suggested CW800-CA should be a safe agent for use in humans.

### Example 7

### Reconstructive Surgery

Surgeons performing reconstructive surgeries needs a safe, simple, minimally invasive means of imaging and determining the anatomical pattern of these key perforating vessels in real-time in the operating room during the planning and execution of flap surgery. In addition, in the postoperative period, the clinician needs a similar means of assessing flap viability and guiding surgical efforts at flap salvage when complications occur.

At the present time, there is little if any intraoperative imaging performed during reconstructive surgery. The reasons for this are multi-fold. First, modalities such as computed tomography (CT), magnetic resonance imaging (MRI), single photon emission computed tomography (SPECT) and positron emission tomography (PET) are impractical and too costly for intraoperative use. Ultrasound is portable, and probes can be rendered sterile, however, it requires contact with the tissue under study, has limited resolution, has limited field of view (FoV), and has limited options with respect to contrast generation. The only other modality available intraoperatively for reconstructive surgery is x-ray angiography. However, angiography requires a dedicated and expensive imaging suite, requires exposure of patient and caregivers to x-rays, and exposes the patient to millimolar concentrations of nephrotoxic iodine. Important for this study, angiography weights all vessels supplying a flap equally, although in cases of perforator artery identification, the ideal imaging technique would weight surface vessels more strongly.

To evaluate the usefulness of NIR agents for such methods, 0.5 mg (14 μg/kg) indocyanine green (ICG) was injected intravenously into a 35 kg Yorkshire pig, and the skin was imaged as described herein.

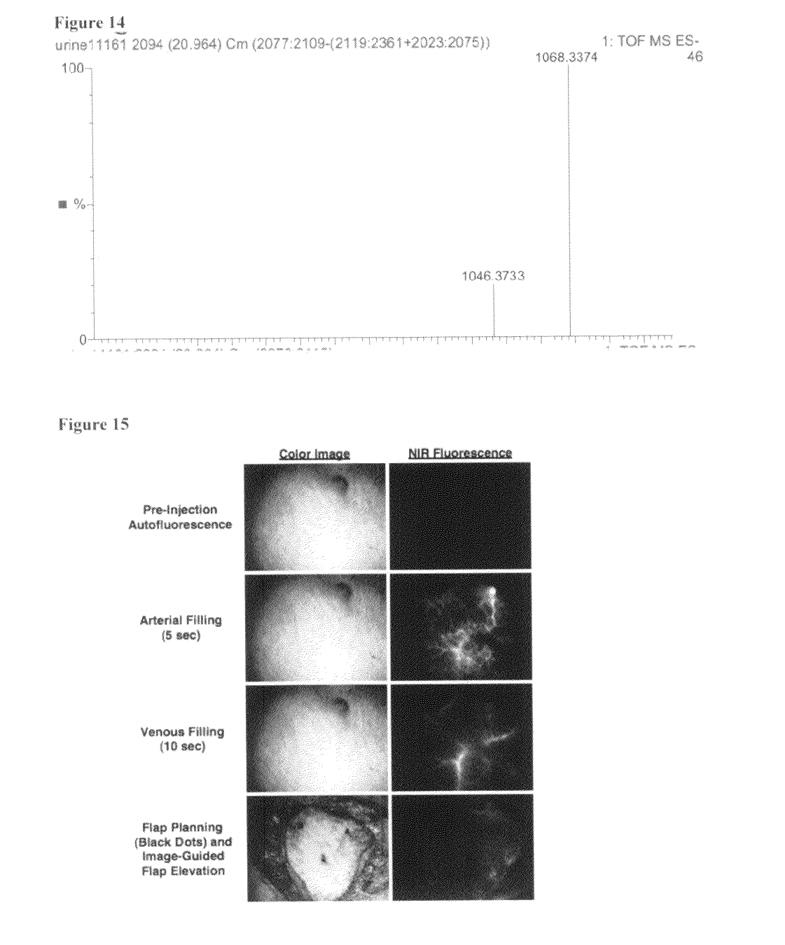

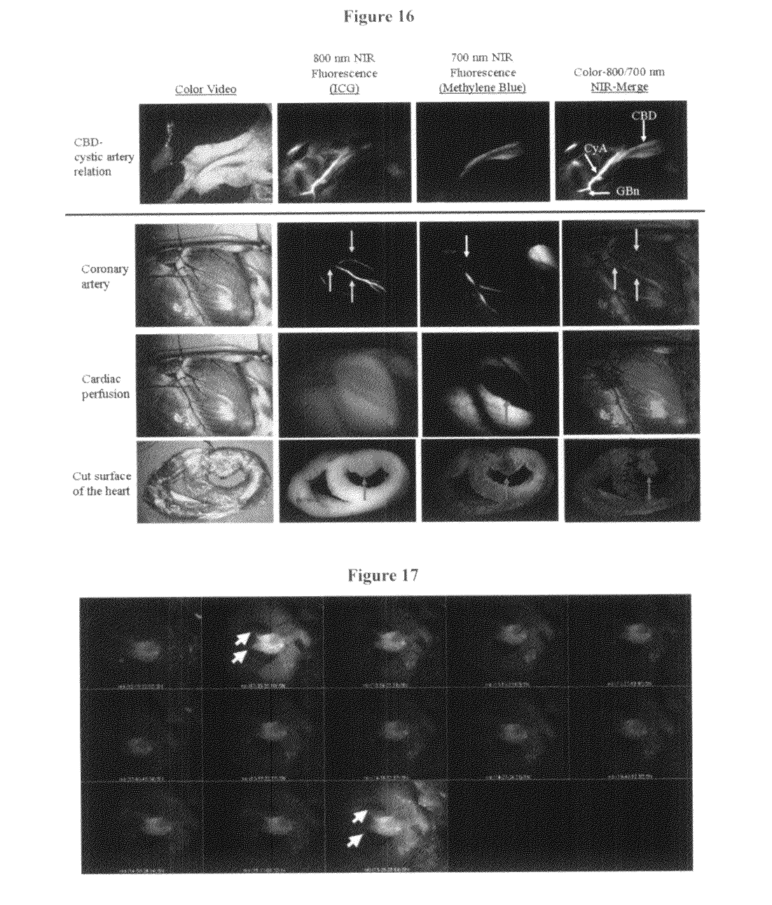

The results are shown in FIG. 15. Shown are pre-injection autofluorescence (top), arterial filling at 5 sec post-injection (second row) and venous filling at 10 sec post-injection. Using these data, the exact location of each perforator is marked on the skin with black marker and the flap is elevated (bottom row). NIR fluorescence images have identical exposure times (67 msec) and normalizations. Cine images were acquired every 200 msec. Note that the nipple in the field (top right) serves as an additional internal control for arterial vs. venous phases. Hence, in real-time, and without requiring any ionizing radiation, the perforating arteries and veins can be identified using NIR fluorescence.

These results indicate that the use of NIR agents, and the intraoperative imaging methods described herein, are useful for flap design, assessment of flap viability, and selection of failing flaps for salvage therapy with fibrinolytics.

### Example 8

### Multi-Wavelength Fluorescence Imaging in Pigs

To evaluate the possibility of multi-wavelength fluorescent imaging, ICG and methylene blue were used.

The results are shown in FIG. 16. 1 mg/kg methylene blue IV injection was followed 30 min after by 0.03 mg/kg ICG IV injection. As can be seen in the first row of images in FIG. 16, the orientation of the common bile duct (CBD), gallbladder neck (GBn), and cystic artery (CyA) is well visualized. NIR camera exposure time was 100 msec.

Next, one of the diagonal branches of the coronary artery (D2) was clamped (white arrow), and then 1 mg/kg of methylene blue was injected intravenously; see the second row of images. Just after releasing the clamp, 0.03 mg/kg ICG was injected intravenously. D2 was detected clearly (yellow arrow). The right-most image in the second row was created by merging the image of the coronary arteriography using methylene blue at clamping D2 and the image using ICG after declamping D2.

After taking arteriography, the ICG was evenly distributed over the entire myocardium (see third row of images, the red pseudocolor in the merged image of cardiac perfusion); however, a perfusion defect could still be detected in 700 nm in the fluorescence image (green pseudocolor) and in the merged image. The area where both dyes are distributed was a weak orange pseudocolor.

15 minutes after ICG injection, the heart was resected and immediately examined; the results are shown in the images in the bottom row. The findings in the third row were confirmed. The NIR camera exposure time was 100 msec.

### Example 9

### Parathyroid Gland Imaging Using Methylene Blue (MB)

To determine whether MB fluorescence emissions could be used to image the parathyroid gland, 2 mg/kg MB was administered over 15 minutes, along with NaHCO₃, prior to surgery. As can be seen in FIG. 17, the parathyroid was indeed visible.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of imaging an anatomical structure selected from the group consisting of vasculature, myocardium, parathyroid gland, a thoracic duct, a biliary tree, a ureter, or a portion thereof, in vivo, the method comprising:
administering to a subject a preparation comprising an invisible light (IL) fluorophore, wherein the administration is systemic or by injection directly into the anatomical structure, and
obtaining an image of IL wavelength emissions from the fluorophore, wherein said image is a representation of the anatomical structure.

2. The method of claim 1, wherein the IL fluorophore is filtered by the kidney into the urine stream, filtered by the liver into the bile stream, or both, and the image is a representation of a biliary tree, a ureter, or a portion thereof.

3. The method of claim 1, comprising:
injecting a preparation comprising an invisible light (IL) fluorophore into a lymph node of a subject, and
obtaining an image of IL wavelength emissions, wherein said image is a representation of the thoracic duct.

4. The method of claim 1, wherein obtaining an image comprises positioning an electronic imaging device adjacent to the structure.

5. The method of claim 1, wherein the image comprises some portion of the subject, and the method includes obtaining a first image of one or more wavelengths of visible light and obtaining a second image of IL wavelength emissions of the IL fluorophore.

6. The method of claim 5, wherein the visible light image and the IL wavelength emissions image are obtained concurrently.

7. The method of claim 1, wherein the preparation is administered intravenously.

8. The method of claim 1, wherein the preparation is administered by direct injection into the anatomical structure to be imaged.

9. The method of claim 1, wherein the anatomical structure is a biliary tree, and the preparation is administered by injection into a portal vein, left hepatic duct, or right hepatic duct.

10. The method of claim 1, wherein the anatomical structure is a ureter, and the preparation is administered by injection into a renal artery, bladder, or ureter.

11. The method of claim 1, wherein the near-infrared fluorophore has an emission wavelength in a range from about 670 nm to about 1,000 nm.

12. The method of claim 1, wherein the near-infrared fluorophore has a structure of the formula: 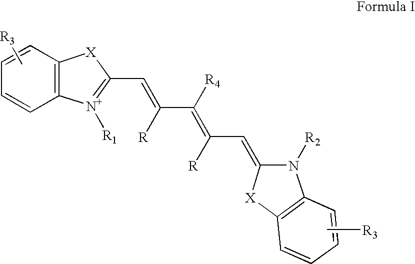
wherein, as valence and stability permit,
X represents C(R)₂, S, Se, O, or NR₅;
R represents H or lower alkyl, or two occurrences of R, taken together, form a ring together with the carbon atoms through which they are connected;
R₁ and R₂ represent, independently, substituted or unsubstituted lower alkyl, lower alkenyl, cycloalkyl, cycloalkylalkyl, aryl, or aralkyl, optionally substituted by sulfate, phosphate, sulphonate, phosphonate, halogen, hydroxyl, amino, cyano, nitro, carboxylic acid, or amide, or a pharmaceutically acceptable salt thereof;
R₃ represents, independently for each occurrence, one or more substituents to the ring to which it is attached, such as a fused ring, sulfate, phosphate, sulphonate, phosphonate, halogen, lower alkyl, hydroxyl, amino, cyano, nitro, carboxylic acid, or amide, or a pharmaceutically acceptable salt thereof;
R₄ represents H, halogen, or a substituted or unsubstituted ether or thioether of phenol or thiophenol;
R₅ represents, independently for each occurrence, substituted or unsubstituted lower alkyl, cycloalkyl, cycloalkylalkyl, aryl, or aralkyl, optionally substituted by sulfate, phosphate, sulphonate, phosphonate, halogen, hydroxyl, amino, cyano, nitro, carboxylic acid, amide; or a pharmaceutically acceptable salt thereof.

13. The method of claim 12, wherein two occurrences of R taken together form a six-membered ring.

14. The method of claim 12, wherein R₁, R₂, and at least one R₃ include sulphonate.

15. The method of claim 1, wherein the near-infrared fluorophore is selected from the group consisting of IRDye78, IRDye80, IRDye38, IRDye40, IRDye41, IRDye700, IRDye800, Cy5.5 and Cy7, or an analog thereof.

16. The method of claim 1, wherein the near-infrared fluorophore is methylene blue (MB).

17. The method of claim 16, wherein the preparation is a solution comprising about 100 nM-100 μM MB.

18. The method of claim 16, comprising administering a sufficient amount of MB to achieve a concentration of about 10-40 μM MB in the structure to be imaged.

19. The method of claim 16, wherein the MB is administered in a total systemic dose of about 0.1 to 10 mg/kg of body weight.

20. The method of claim 16, wherein the MB is administered in a total systemic dose of about 1 mg/kg of body weight.

21. The method of claim 16, wherein the MB is administered in a total systemic dose of less than 7.5 mg/kg of body weight.

22. A method of imaging an anatomical structure in vivo, the method comprising:
administering to a subject a preparation comprising methylene blue (MB) such that it appears in the anatomical structure, wherein the preparation is administered systemically or by direct injection into the structure, and
obtaining an image of invisible light wavelength emissions, wherein said image is a representation of the anatomical structure.

23. The method of claim 22, wherein the preparation comprises from 0.1 to 10% MB.

24. The method of claim 22, wherein the preparation comprises about 100 nM-100 μM MB.

25. The method of claim 22, comprising administering a sufficient amount of MB to achieve a concentration of about 10-40 μM MB in the structure to be imaged.

26. The method of claim 22, wherein the MB is administered in a total systemic dose of about 0.1 to 10 mg/kg of body weight.

27. The method of claim 22, wherein the MB is administered in a total systemic dose of about 1 mg/kg of body weight.

28. The method of claim 22, wherein the MB is administered in a total systemic dose of less than 7.5 mg/kg of body weight.

29. The method of claim 22, wherein the structure is vasculature, biliary tree, thoracic duct, ureters, or a portion thereof.

30. The method of claim 22, wherein the tissue or organ has high uptake of MB.

31. The method of claim 22, wherein the tissue or organ is myocardium or parathryoid gland.

32. A method of imaging first and second anatomical structures in vivo, the method comprising:
administering to a subject a preparation comprising methylene blue (MB) such that it appears in the first anatomical structure, and
obtaining a first image of invisible light wavelength emissions of the MB, wherein said image is a representation of the first anatomical structure;
administering to the subject a preparation comprising a second invisible light fluorophore (ILF) with an emission wavelength of at least about 780 nm such that the second ILF appears in the second anatomical structure, and
obtaining a second image of the invisible light emissions of the second ILF, wherein the image of the invisible light emissions of the second ILF is a representation of a second anatomical structure.

33. The method of claim 32, wherein the anatomical structure represented by the first image is vasculature, biliary tree, thoracic duct, ureters, heart, parathyroid glands, or a portion thereof.

34. The method of claim 32, wherein the anatomical structure represented by the second image is vasculature, biliary tree, thoracic duct, ureters, heart, parathyroid glands, or a portion thereof, and is different from the anatomical structure represented by the first image.

35. The method of claim 32, wherein the second ILF is indocyanine green (ICG).

36. The method of claim 32, wherein the second ILF is the carboxylic acid of IRDye™800CW.

37. The method of claim 32, further comprising obtaining a visible light image of the structures.

38. The method of claim 37, wherein the first image, second image, and visible light image, are all obtained concurrently.

39. A method of imaging the vasculature of a tissue for evaluation of flap design desirability, assessment of flap viability, or determination of suitability of a failing flap for salvage therapy with fibrinolytics, the method comprising:
administering to the subject a preparation comprising an invisible light (IL) fluorophore, and
obtaining an image of invisible light wavelength emissions, wherein said image is a representation of the vasculature of the tissue, and wherein the vasculature of the tissue indicates the desirability of the flap design, the viability of the flap, or the suitability of a failing flap for salvage therapy with fibrinolytics.
